(19) United States
(12) Patent Application Publication
(10) Pub. No.: US 2012/0296187 A1
(43) Pub. Date: Nov. 22, 2012

(54) Devices and Methods for Obtaining Analyte Sample

(75) Inventors: Timothy P. Henning, Vernon Hills, IL (US); John Bhogal, San Francisco, CA (US); Andrew H. Naegeli, Walnut Creek, CA (US)

(73) Assignee: Abbott Diabetes Care Inc.

(21) Appl. No.: 13/453,448

(22) Filed: Apr. 23, 2012

Related U.S. Application Data
(60) Provisional application No. 61/481,125, filed on Apr. 29, 2011.

(51) Int. Cl. A61B 5/1473; B23P 17/04

(52) U.S. Cl. 600/347; 29/592; 29/527.1; 29/557

(57) ABSTRACT

Devices, systems and methods for accessing bodily fluid beneath the skin surface by abrading the skin, whereby such bodily fluid may be extracted for ex vivo analysis or whereby an in vivo analyte sensor is implanted at the abrasion site.

### CROSS-REFERENCE TO RELATED APPLICATIONS

Pursuant to 35 U.S.C. §119(e), this application claims priority to U.S. Provisional Patent Application No. 61/481,125 filed on Apr. 29, 2011, the disclosure of which is herein incorporated by reference in its entirety.

### RELATED MATTERS

The present application is related to U.S. provisional patent application No. 61/480,883 filed on Apr. 29, 2011 and U.S. patent application titled “Methods of Collecting and Analyzing Sample” (Attorney Docket No. ADCI-236), concurrently filed herewith, and assigned to the assignee of the present application, and the disclosure of which is incorporated herein by reference for all purposes.

### BACKGROUND

Analyte detection in physiological fluids, e.g., blood, blood-derived products or interstitial fluid, is of ever increasing importance to today's society. Analyte detection assays find use in a variety of applications, including clinical laboratory testing, home testing, etc., where the results of such testing play a prominent role in the diagnosis and management of a variety of disease conditions. Analytes of interest include glucose, ketones, lactate, oxygen, cholesterol (including HDL, LDL, and/or triglycerides), hemoglobin A1C, and the like. The monitoring of glucose is particularly important for diabetes management, e.g., in many instances, diabetics must determine when insulin is needed to be administered to reduce glucose levels in their bodies or when additional glucose is needed to raise the level of glucose in their bodies.

A variety of analyte detection protocols and devices for both clinical and home use have been developed. Historically, blood glucose and other bodily analyte measurements were invasive. Such measurements were generally made by withdrawing a blood sample and measuring the desired analyte within the blood or plasma. Blood samples were typically withdrawn by inserting a needle into a major artery or, more commonly, a vein. Such direct vascular blood sampling employed by these early methods had several limitations, including pain, hematoma and other bleeding complications, and infection. In addition, due to the vascular damage resulting from the needle puncture, sampling could not be repeated on a routine basis. Finally, it was extremely difficult for patients to perform a direct vascular puncture on themselves.

A more recent technique that has been developed to overcome some of the disadvantages associated with the above protocols is to collect a blood sample by cutting or lancing the skin and the subcutaneous tissue, including the small, underlying blood vessels, to produce a localized bleeding on the body surface. The accessed blood is then collected onto a reagent test strip onto which the blood sample is placed. The fingertip is the most frequently used site for this method of blood collection due to the large number of small blood vessels located therein. This “in vitro” or “ex vivo” method has the significant disadvantage of being very painful because subcutaneous tissue of the fingertip has a large concentration of nerve endings. It is not uncommon for patients who require frequent monitoring of an analyte to avoid having their blood sampled. With diabetics, for example, the failure to frequently measure their glucose level on a prescribed basis results in a lack of information necessary to properly control the level of glucose. This technique of blood sampling also runs the risk of infection and the transmission of disease to the patient, particularly when done on a high-frequency basis. These drawbacks are further exacerbated by the fact that there is a limited amount of skin surface that can be used for the frequent sampling of blood without forming thick calluses.

In recent years, a variety of temporarily implantable or “in vivo” sensors have been developed for detecting and/or quantifying analytes in a patient's body fluid, such as blood or interstitial fluid. Such in vivo analyte sensors may be fully or partially implanted below the epidermis in a blood vessel or in the subcutaneous tissue of a patient for direct and constant contact with blood or other extra-cellular fluid, such as interstitial fluid, wherein such sensors can be used to obtain periodic and/or continuous analyte readings over a period of time. Certain transcutaneous analyte sensors have an electrochemical configuration in which the implantable portion of these sensors includes exposed electrodes and chemistry that react with a target analyte. At an externally located proximal end of the sensor are exposed conductive contacts for electrical connection with a sensor control unit which is typically mountable on the skin of the patient. Using such in vivo sensor systems, analyte testing is performed with the in vivo analyte sensor so-positioned (e.g., transcutaneously positioned or wholly implanted), and analyte-related signals (e.g., current or the like) are obtained continuously over a period of time, as opposed to at discrete time points as with in vitro systems that use a biological sample expressed at a given time from a user and applied to an analyte test strip exterior to the body. The components of in vivo analyte systems can vary, but may include an in vivo analyte sensor, a sensor control unit configured for direct or indirect electrical contact with the in vivo sensor, and a receiver unit or monitor to receive communication from the sensor control unit, e.g., wirelessly by radio frequency (RF), infrared (IR), and the like, or with a wired connection using an electrical cable. An example of an in vivo glucose monitoring system includes the FreeStyle Navigator® continuous glucose monitoring system from Abbott Diabetes Care Inc. of Alameda, Calif. Examples of sensors and associated analyte monitoring systems can be found in, for example, but not limited to, U.S. Pat. Nos. 6,134,461; 6,175,752; 6,284,478; 6,560,471; 6,579,690; 6,746,582; 6,932,892; 7,299,082; 7,381,184; 7,618,369 and 7,697,967; and U.S. Patent Application Publication Nos. 2008/0161666, 2009/0054748, 2009/0247857 and 2010/0081909, the disclosures of which are incorporated by reference herein.

To monitor an analyte with an in vivo sensor, the sensor must first be appropriately positioned at a location on or in a user's body. Positioning sensors in this way has its challenges. Conventionally, needles, or needle-like devices, i.e., sharps, are used to create a wound in the skin to allow for the transcutaneous insertion of an analyte sensor. For example, early applications involved the use of a sharp, steel needle that also served as a working electrode of the sensor. The sharp needle/electrode was used to puncture the skin to create a wound into which the needle/sensor resided for the duration of analyte monitoring. In another approach, metal needles or sharps, often referred to as introducers, are used, but in combination with a separate analyte sensor in which the two are releasably coupled together. In certain configurations, the introducer needle has a slotted or hollow configuration in which a distal portion of the sensor is slidably carried. The needle is used to pierce the skin and to create a wound through the skin to the target site while carrying the sensor to the desired implantation site, e.g., subcutaneous site, while protecting the sensor from being damaged during insertion. The needle is then removed from the wound and the sensor is left positioned in the user. Examples of such insertion devices are disclosed in U.S. Pat. No. 7,381,184.

Whether integrated within the sensor structure or used as a separate component to facilitate sensor insertion, needles or sharps have their drawbacks, including further complicating the sensor positioning process, particularly when such devices are configured to push the metal insertion needle into the skin, decouple from the sensor, and retract the needle back out of the skin, all of which require precise timing and manipulation. Such mechanics, whether used manually, semi-manually or automatically, add costs to analyte monitoring systems. Other drawbacks of needles and sharps for facilitating the temporary implantation of in vivo sensors are that they can be painful and cause bleeding, which can be disturbing to the user, and can result in inaccurate readings, at least until the bleeding and/or body response to the bleeding resolves.

In addition to drawbacks of needles and sharps, there are drawbacks to the subcutaneous placement and implantation of analyte sensors themselves. Such subcutaneous implantation produces both short-term and longer-term biochemical and cellular responses. For example, it has been found that during the initial 12 to 24 hours of sensor operation (after implantation), an analyte sensor's sensitivity may be relatively low—a phenomenon sometimes referred to as “early sensor attenuation” (ESA). Even subsequent to this initial period of ESA, spurious low readings or drop outs may be caused by the presence of blood clots, also known as “thrombi”, that form as a result of the transcutaneous insertion of the sensor. Such clots exist in close proximity to the subcutaneous sensor and have a tendency to “consume” the target analyte, such as glucose, at a high rate, thereby lowering the local analyte concentration. It may also be that the implanted sensor constricts adjacent blood vessels, thereby restricting analyte delivery or flux to the sensor site. Still yet, as part of the immune response, a foreign body capsule may develop around the implanted sensor which may reduce the flux of analyte to the sensor, i.e., may reduce the sensitivity or accuracy of the sensor function. An in vivo glucose sensor, for example, with lower than normal sensitivity may report blood glucose values lower than the actual values, thus potentially underestimating hyperglycemia, and triggering false hypoglycemia alarms.

In order to compensate for such biochemical and cellular effects on sensor sensitivity, the sensor may require frequent recalibration over the course of the sensor's implantation period. This is often accomplished in the context of continuous glucose monitoring devices by using a reference value after the sensor has been positioned in the body, where the reference value most often employed is obtained by use of a blood glucose test strip for which a blood sample is obtained by means of a finger stick, which is inconvenient and can cause significant discomfort to the patient. Besides the technical aspects of recalibration and the burden upon the patient to recalibrate an implanted sensor, putting calibration in the hands of a patient presents safety and quality issues.

The extent of the immune response presented by implantable sensors, and the resulting sensor calibration and performance issues, are exacerbated by the size of the implantable portion of the sensor, often referred to as the “sensor tail”, and/or by the sensor introducer, i.e., the sharp or needle. A relatively large outer diameter of the sensor tail and/or introducer results in a more traumatic transcutaneous introduction which, in turn, produces a greater immune response to the sensor, as well as increased pain and discomfort felt by the patient. Accordingly, an objective of sensor manufacturers has been to minimize sensor and introducer size while providing a highly reliable and reproducible product. Such sensor miniaturization, however, requires extremely precise fabrication processes and equipment, which increase manufacturing costs. For example, modifying an introducer needle or sharp, e.g., creating the longitudinal slot or slit within it, to allow it to accept a sensor may require use of very expensive laser equipment. Reducing introducer size necessarily requires reducing sensor size which, without precision fabrication and the use of highly expensive materials, will sacrifice sensor quality and reliability. Because the surface area of the electrodes or conductive traces on these miniaturized sensors is so limited, the conductive material itself must be highly conductive and very reliable, which is why many currently available implantable sensors are made with gold or platinum conductive traces, further adding to the cost of these sensors and their associated monitoring systems.

Accordingly, of interest are devices and methods for in vivo analyte monitoring which overcome or minimize the drawbacks of conventional analyte monitoring devices and methods. For example, it would be greatly advantageous to provide in vivo sensors which do not require a sharp or an introducer needle for transcutaneous implantation, nor are themselves sharply pointed—in order to reduce pain, minimize bleeding and avoid trauma to the target implantation site, and, thus, minimize the corresponding physiological response that may interfere with the sensors' sensitivity and/or accuracy. It would be additionally beneficial to provide methods of transcutaneously accessing bodily fluids, such as interstitial fluid, for either in vivo or ex vivo analyte testing in a manner that avoids and minimizes these same drawbacks.

### SUMMARY

The subject devices, systems and methods are configured to access the desired bodily fluid, i.e., blood or interstitial fluid (ISF), in a target skin layer, i.e., dermis, epidermis or stratum corneum, or in subcutaneous tissue. In various applications of the present disclosure, particularly when the preferred target bodily fluid is ISF rather than blood, the skin is accessed to a depth less than the dermis layer, i.e., to within the epidermis or stratum corneum, thus avoiding blood vessels. Such skin layer and/or bodily fluid access is achieved with the present disclosure without the use of a sharp or pointed implement or one that is otherwise needle-less or sharp-less, i.e., without cutting, lancing or pointed edges or ends. Instead, access is enabled by the removal of skin or skin cells by abrasion or abrasive action provided by an abrasion device. The abrasion may be carried out to the extent to cause ISF or blood to be expressed or to be expressible at a target location on the skin for subsequent extraction for ex vivo analysis of one or more analytes therein. Such ex vivo analysis may be conducted, for example, by means of a laboratory assay or with the use of an analyte test strip and handheld meter. Alternatively or additionally, such abrasion may be carried out to create an opening extending to a maximum depth at or within a targeted skin layer, which opening is sufficient to enable the subsequent needle-less or sharp-less penetration of a blunt sensor to within subcutaneous tissue where the sensor is used for the temporary in vivo monitoring of one or more analytes contained within the bodily fluid existing within the targeted subcutaneous tissue. As such, the present disclosure also provides for systems and methods which utilize the subject abrasion devices for accessing a target skin layer or a desired bodily fluid as well as additional devices for either extracting bodily fluid from the skin or for positioning an in vivo sensor within the skin, or both.

One exemplary method of the present disclosure is directed to transcutaneously accessing bodily fluid. At least one skin attachment member is affixed to the skin surface adjacent at least one target access site and an abrasion device is mounted thereon. The abrasion device includes an abrasion head having a blunted skin-contacting surface which, when mounted to the skin attachment platform, is aligned above a targeted bodily fluid access site. The skin is then abraded to a selected depth, which may be accomplished by adjusting the height of the platform to selectively control the depth of abrasion by the abrasion device. Depending on the application, and on the fluid to be accessed, such as ISF, the depth of abrasion may not be greater than the epidermis or the stratum corneum. The abrasion device may be removed from the at least one skin attachment platform, and repositioned and remounted to the skin attachment member as needed to abrade any number of target fluid access sites. After the desired number of fluid access sites is abraded, the target fluid is extracted from the skin, which extraction may be facilitated by any one or more of a number of methods including, but not limited to, applying negative pressure to the skin, mechanically stretching the skin, applying capillary action to the skin, heating the skin, and applying a chemical at the at least one bodily fluid access site.

Another method of the present disclosure is directed to transcutaneously positioning a sensor and includes abrading a target site on a skin surface to a selected depth, such as by the method described above, and then positioning an implantable portion of a sensor into the skin through the abraded target site. In certain methods, the selected depth is not greater than the epidermis, and in others, the selected depth is not greater than the stratum corneum. Abrading the skin may be accomplished by use of an abrasion device having a blunted skin-contacting surface. Prior to abrading the target site, a platform may be affixed to the skin surface adjacent the target site and the abrasion device mounted thereto. The height of the platform may be selectively adjusted to control the depth of abrasion into the skin. Additionally, the speed of the abrasion may be selectively controlled. The sensor, which can have a blunted distal end, may be positioned to a depth greater than that made by the abrading, and may even extend into subcutaneous tissue. A sensor control unit may be mounted to the platform and to the sensor after transcutaneously positioning the sensor.

The present disclosure is also directed to transcutaneously implantable sensors. In certain embodiments, the subject sensors include a proximal portion configured for positioning above a skin surface and for connecting with a sensor control unit, and a distal portion having a blunted distal end configured for penetrating through an opening or an otherwise compromised stratum corneum layer of the skin. An implantable length of the distal portion of the sensor is provided which is sufficient to extend into the target skin layer or to within a skin layer containing the target fluid, e.g., ISF, blood, to be accessed. For example, the implantable length may extend into subcutaneous tissue when the sensor is operatively transcutaneously positioned.

Certain embodiments of the subject sensors are formed of a housing made of non-conductive material. The implantable length of the distal portion of the housing includes at least one aperture therein to expose a sensing component, e.g., electrode, electrochemical sensing material, etc., housed within the distal portion. The at least one electrode extends from within the distal portion to within the proximal portion where it is positioned for operable coupling to an electronics unit, such as a sensor control unit which is mountable to the proximal portion of the sensor.

The subject sensors may be fabricated using various techniques and processes. In one such process, at least one conductor is provided and the non-conductive sensor housing is formed about the at least one conductor. To accomplish such, the conductor is provided or formed on an insulating core and the sensor housing is then molded about the insulating core. Alternatively, the sensor housing may be formed or molded first and the at least one conductor positioned within the molded housing thereafter. This may be accomplished by forming one or more channels with the housing, from the distal portion to the proximal portion, and then providing the conductor or conductive material within the at least one channel. At least one aperture is provided within the implantable portion of the distal portion of the housing to expose the at least one conductor where the aperture is formed as part of molding the housing or is otherwise created subsequently within the molded housing structure.

The present disclosure also includes systems for abrading the skin surface to access a target fluid as well as systems for transcutaneously positioning a sensor. In one embodiment, the system includes an abrasion device and skin attachment platform. The abrasion device includes a base unit and a rotatable shaft having an abrasion head provided at a distal end thereof. The base unit houses a component for powering the device and rotating the shaft, which rotation may be at a selectable speed. The abrasion head, in some embodiments where it is an objective to minimize pain and bleeding, is made of material effective for abrading the stratum corneum but otherwise ineffective in abrading the epidermis. The skin attachment platform includes a bottom surface configured for placement on a skin surface, such as by an adhesive layer or the like, and a top surface configured for releasably mounting with the base unit. The skin attachment platform may further include components for adjusting the depth to which the abrasion head is extendable below the bottom surface of the skin attachment platform. In some embodiments, the depth adjustment components are height-adjustable posts provided on the top surface of the skin attachment platform. In other embodiments, the depth adjustment components include at least one removable spacer layer positionable on the top surface of the skin attachment platform, where a plurality of abrasion heads may be interchangeable but where each may be unique in one or more of shape, size and abrasiveness.

In certain embodiments, the skin attachment platform may include at least two structures or members which are positionable about a target abrasion site and function cooperatively to enable selective positioning of the abrasion device.

These and other embodiments, objects, advantages, and features of the present disclosure will become apparent to those persons skilled in the art upon reading the details of the present disclosure as more fully described below.

### BRIEF DESCRIPTION OF THE DRAWINGS

The present disclosure is best understood from the following detailed description when read in conjunction with the accompanying drawings. It is emphasized that, according to common practice, the various features of the drawings are not to scale. On the contrary, the dimensions of the various features are arbitrarily expanded or reduced for clarity. Included in the drawings are the following figures:

FIGS. 1A and 1B are perspective and longitudinal cross-sectional views, respectively, of a skin abrasion device according to certain embodiments of the present disclosure;

FIG. 2A is a perspective view of an embodiment of a skin attachment member of the present disclosure; FIG. 2B is a cross-sectional view of another embodiment of a skin attachment member of the present disclosure; and FIG. 2C is a cross-sectional view of another skin attachment of the present disclosure;

FIG. 3A shows a perspective view of an embodiment of an in vivo analyte sensor according to the present disclosure; FIG. 3B shows a longitudinal cross-sectional view of the sensor of FIG. 3A; FIG. 3C shows a sectional view of the sensor of FIG. 3B taken along line C-C; and FIG. 3D shows another sectional view of the sensor of FIG. 3B taken along line D-D;

FIGS. 4A-4C illustrate a process of the present disclosure of abrading the skin to create one or more openings in the skin surface using a skin attachment member and mechanical abrasion device of the present disclosure; and FIGS. 4D-4F illustrate methods of the present disclosure for transcutaneously positioning the analyte sensor of FIG. 3A into the one or more skin openings for performing in vivo analyte monitoring therewith; and

FIGS. 5A and 5B are top planar views of other embodiments of skin attachment devices of the present disclosure configured to enable the selective formation of multiple openings in the skin surface with the skin abrasion devices of the present disclosure and/or using the method of FIGS. 4A-4C.

### DETAILED DESCRIPTION

Before the present disclosure is further described, it is to be understood that this present disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Where a range of values is provided, it is understood that each intervening value, to the tenth of the unit of the lower limit unless the context clearly dictates otherwise, between the upper and lower limit of that range and any other stated or intervening value in that stated range, is encompassed within the present disclosure. The upper and lower limits of these smaller ranges may independently be included in the smaller ranges, and are also encompassed within the present disclosure, subject to any specifically excluded limit in the stated range. Where the stated range includes one or both of the limits, ranges excluding either or both of those included limits are also included in the present disclosure.

Unless defined otherwise, all technical and scientific terms used herein have the same meaning as commonly understood by one of ordinary skill in the art to which this present disclosure belongs. Although any methods and materials similar or equivalent to those described herein can also be used in the practice or testing of the present disclosure, exemplary methods and materials are now described. All publications mentioned herein are incorporated herein by reference to disclose and describe the methods and/or materials in connection with which the publications are cited.

As used herein and in the appended claims, the singular forms “a,” “an,” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

The publications discussed herein are provided solely for their disclosure prior to the filing date of the present application. Nothing herein is to be construed as an admission that the present disclosure is not entitled to antedate such publication by virtue of prior present disclosure. Further, the dates of publication provided may be different from the actual publication dates which may need to be independently confirmed.

To overcome the disadvantages of conventional analyte monitoring or testing techniques that are associated with a high degree of pain, bleeding and interfering physiological responses, as described above, the present disclosure is directed in part to controlling and/or minimizing the skin depth at which bodily fluid is accessed for obtaining, extracting or retrieving the desired bodily fluid, i.e., blood or interstitial fluid (ISF), for ex vivo analyte testing, or is accessed for the in vivo detection and monitoring of one or more analytes.

Above the subcutaneous tissue, there are several layers of skin. The stratum corneum is the outermost layer which is generally between about 10 and about 50 cells thick and extends between about 10 μm to about 20 μm from the exterior surface of the skin. These cells, called keratinocytes, are filled with bundles of cross-linked keratin and keratohyalin surrounded by an extracellular matrix of lipids. Below the stratum corneum is the viable epidermis, which is between about 50 μm and about 100 μm thick. The viable epidermis contains no blood vessels, and it exchanges metabolites by diffusion to and from the dermis, which is the skin layer beneath the viable epidermis. The dermis is between about 1 mm and about 3 mm thick and contains blood vessels, lymphatics and nerves.

The subject devices, systems and methods are configured to access the desired bodily fluid, i.e., blood or interstitial fluid, in a target skin layer, i.e., dermis, epidermis or stratum corneum, or in subcutaneous tissue. In many embodiments and methods of the present disclosure, particularly when the preferred target bodily fluid is ISF rather than blood, the skin is accessed to a depth less than the dermis layer, i.e., to within the epidermis or stratum corneum, thus avoiding blood vessels. Access at a depth less than the dermis layer also avoids nerve endings, thereby minimizing pain felt by the patient. It is noted that the terms “access” and “accessing”, when used herein in the context of access to or accessing a target skin layer or a bodily fluid within the skin, include reaching or contacting or the like.

Such skin layer and/or bodily fluid access is achieved with the present disclosure without the use of a sharp or pointed implement, i.e., without cutting, lancing or pointed edges or ends. Instead, in certain embodiments, access is enabled by the removal of skin or skin cells by abrasion or abrasive action provided by an abrasion device. The abrasion may be carried out to the extent to cause ISF or blood to be expressed or to be expressible at a target location on the skin for subsequent extraction for ex vivo analysis of one or more analytes therein. Such ex vivo analysis may be conducted, for example, by means of a laboratory assay or with the use of an analyte test strip and handheld meter. Alternatively or additionally, such abrasion may be carried out to create an opening extending to a maximum depth at or within a targeted skin layer, which opening is sufficient to enable the subsequent needle-less or sharp-less penetration of a blunt sensor to within subcutaneous tissue where the sensor is used for the temporary in vivo monitoring of one or more analytes contained within the bodily fluid existing within the targeted subcutaneous tissue. As such, the present disclosure also provides for systems and methods which utilize the subject abrasion devices for accessing a target skin layer or a desired bodily fluid as well as additional devices for either extracting bodily fluid from the skin or for positioning an in vivo sensor within the skin, or both.

For applications involving the extraction of bodily fluid, e.g., ISF, from the skin for ex vivo analysis, the subject systems may include, in addition to a skin abrasion device as described herein, a fluid extraction device, which may employ one or more components or types of energy for expressing the bodily fluid from the skin. Alternatively, the abrasion and fluid extraction functions may be integrated into a single device. In either configuration, the fluid extraction device/function may employ or be provided by negative pressure (i.e., suction, vacuum) or mechanical stretching of the skin to pool the fluid at the skin surface, by capillary collection to draw out the fluid, by heat to increase the rate of fluid flow near the skin surface, and/or by chemical means, such as histamine, to cause a physiological response to increase perfusion under and within the target site. Once extracted from the target site, the extracted ISF or the fluid may be used for assaying one or more analytes, e.g., glucose. Examples of such extraction techniques are described in, for example, but not limited to, U.S. Pat. No. 6,155,992, which is herein incorporated by reference in its entirety.

For applications involving the positioning or placement of a sensor within the skin for the in vivo measurement or monitoring of one or more analytes within bodily fluid contained within the skin, the subject systems may include, in addition to a skin abrasion device as described herein, a sensor positioning device in which neither the sensor positioning device nor the sensor to be positioned therewith includes a sharp or point for facilitating sensor placement within the subcutaneous tissue. In certain embodiments, the analyte sensor may include a blunted distal or leading end for placement within the skin through a compromised stratum corneum, e.g., within an abraded opening with the stratum corneum, which opening may be created by the subject abrasion device.

With either ex vivo or in vivo analyte measurement applications, the subject skin abrasion devices may be used with a platform or the like which is attachable to the skin surface and which functions to stabilize movement of the abrasion device during use and/or to control the depth of abrasion. In in vivo applications, the subject skin attachment member may be configured to further function to control the depth of penetration of the analyte sensor within the skin and, in certain applications, to subsequently function as a mounting unit for coupling the implanted sensor to an analyte monitoring control or electronics unit.

With reference to the figures, exemplary embodiments of the subject devices, systems and methods are now described.

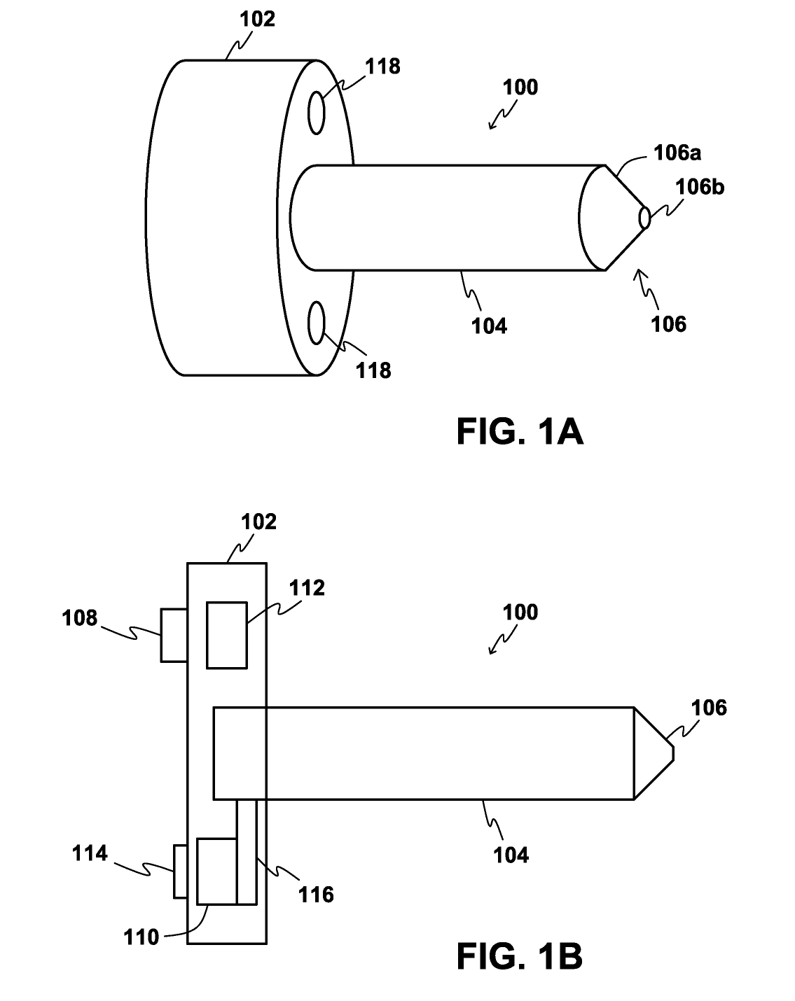

FIGS. 1A and 1B illustrate an embodiment of a skin abrasion device 100 of the present disclosure. Abrasion device 100 includes base unit 102 attached or attachable to rotatable shaft 104. Rotatable shaft 104 terminates at a distal end in abrasion head or grindstone 106. In the illustrated embodiment, abrasion head 106 has a frustum conical shape having a distally tapered tip portion 106a and distally-facing surface 106b, but may have any suitable configuration including rounded, hemispherical, etc. Skin-contacting surface 106b may itself have any suitable shape, where in many embodiments it is blunted or sharpless, i.e., flattened, rounded, or the like. Grindstone 106, including either or both of tapered tip portion 106a and distally-facing surface 106b, and, optionally, any portion of shaft 104, includes a roughened texture capable of removing the surface of the skin without expressing blood or expressing very little blood when abrasion head 106 is engaged with the skin and rotated.

A variety of materials may be used for some or all of the surface of abrasion head 106, as well as shaft 104, including, but limited to, carbon materials such as graphite, diamond, silicon carbide, and other materials such as or aluminum oxide and sand paper. In certain embodiments, some or all of shaft 104 and/or abrasion head 106 is a diamond coated cylindrical device. In some embodiments, some or all of shaft 104 and/or abrasion head 106 are coated with abrasive grains bound in a synthetic resin, wherein the abrasive grains may include diamond and Cr₂O₃, e.g., more than 50% diamond and less than 5% Cr₂O₃, where the grain size may range from D181 (in accordance with the FEPA standard, average particle diameter=181 μm) to D2 (average particle diameter=2 μm). The subject grindstones or abrasion heads made of these materials are advantageous for targeting ISF while minimizing bleeding as they are very effective at abrading away the dry, hard stratum corneum, but are less effective at abrading the epidermis due to the additional moisture contained therein.

Abrasion head 106 may be permanently or removably connected to shaft 104 which itself may also be removable from the base unit 102. As such, abrasion device 100 may be usable with a plurality of interchangeable configurations of abrasion heads 106 and/or shafts 104, where each configuration has a unique or particular size, shape and features, e.g., abrasiveness. A particular configuration would be selected for use based on the body location to be abraded, e.g., the arm, leg, torso, etc., and on the condition of a particular user's skin. Also, two or more abrasion heads 106 having varying configurations may be used at a single abrasion site to progressively abrade the skin until the desired abrasion depth has been achieved. Abrasion head 106 with or without shaft 104 may be re-useable or configured as a disposable, single-use component. If re-useable, abrasion head 106 is preferably capable of being at least disinfected or sterilized.

The diameter of abrasion head 106 may be uniform over its length, or it may be non-uniform. In the illustrated embodiment having a tapered configuration, for example, the cross-sectional diameter of the widest portion, e.g., at the interface with shaft 104, of abrasion head 106 may range from about 0.5 mm to about 5 mm, and the cross-sectional diameter of the narrowest portion, e.g., distally facing surface 106b, may range from about 0.1 mm to about 2 mm.

Referring back to FIG. 1B, base 102 houses mechanisms for rotating shaft 104. A trigger mechanism or actuator switch 108, e.g., in the form of a button or the like, is provided on base 102 for selectively activating motor 110 which, in turn, is operatively coupled to drive mechanism 116 which is operatively coupled to shaft 104 to rotate it about its longitudinal axis. The coupling mechanism (not shown) between drive mechanism 116 and shaft 104 may be any suitable coupling means, e.g., rack and pinion gear, a pulley mechanism, a releasable spring, or the like, for translating the movement of motor 110 into the rotation of shaft 104. A power source 112, e.g., a battery such as a rechargeable battery, is operatively connected to motor 110 to provide power to motor 110. Base 102 may have a closeable battery access opening (not shown) adjacent battery 112 for replacing battery 112 as needed. In certain embodiments, a mechanical abrasion device may include a spring that may be wound by a user every time that an abrasion action is required. The spring would replace motor 110 and battery 112 as a source of power to rotate the shaft. In certain embodiments, abrasion device 100 may include means of controlling, i.e., increasing or decreasing or varying, the speed of motor 110, for which a speed control actuator 114 is provided on base 102 and operatively connected to motor 110 to selectively control the speed of rotation of shaft 104 and abrasion head 106. In other embodiments, the rotational speed of the device is fixed, i.e., is pre-set and not selectable or changeable post-manufacture. Suitable motor speed control means may include, for example a variable resistor or the like between battery 112 and motor 110.

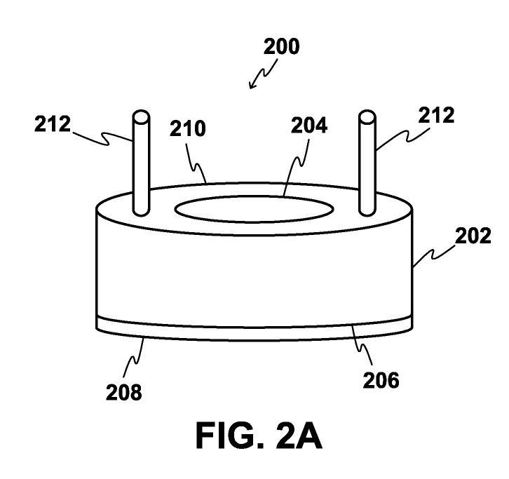

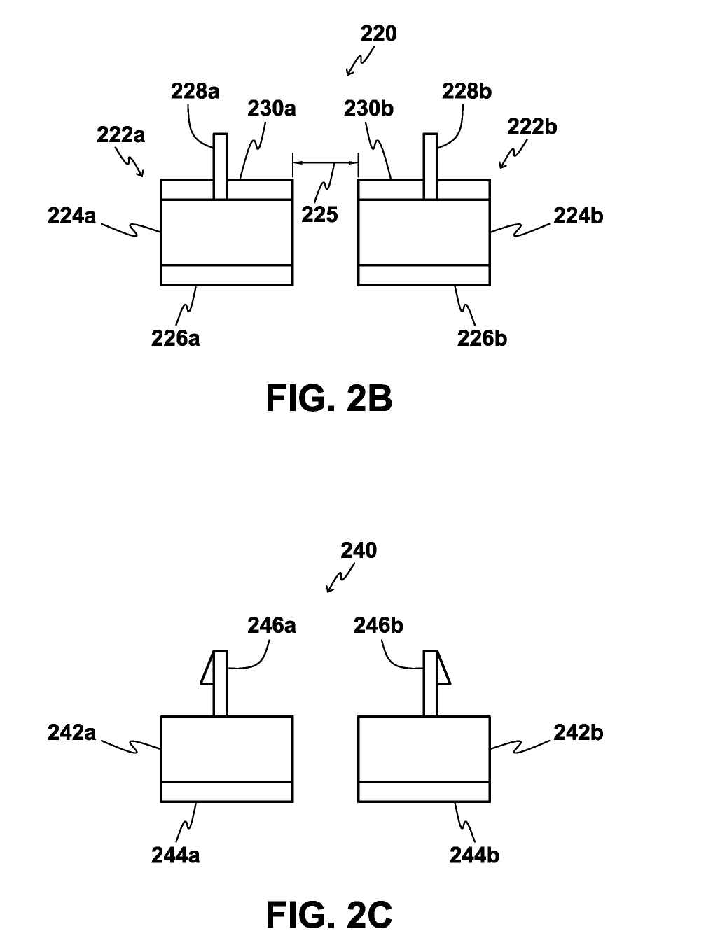

While abrasion device 100 may be a standalone device, to facilitate depth control, i.e., the depth of penetration of abrasion head 106 into the skin surface, and to better stabilize the device during use, abrasion device 100 may be configured to be used with a skin attachment platform, such as illustrated in FIGS. 2A-2C. For example, skin attachment platform 200 of FIG. 2A may be in the form of an annular ring 202 in which a central opening 204 is configured and dimensioned to receive shaft 104 of abrasion device 100 of FIGS. 1A and 1B (as well as an analyte sensor for in vivo analyte monitoring applications, discussed in greater detail below). The bottom or skin-contacting surface 206 of skin attachment ring 202 is configured to engage with and attach to the skin surface, such as by means of a strap member (not shown) or an adhesive layer or pad 208. The top surface 210 of skin attachment ring 202 is configured to engage with and attach to base 102 of abrasion device 100, such as by one or more alignment features 212, herein shown in the shape of posts, that enable attachment of abrasion device 100 by way of mating engagement with one or more corresponding alignment features 118 within base unit 102 of abrasion device 100. As shown in FIG. 1A, alignment features 118, herein in the form of holes, may be provided at locations within base 102 of abrasion device 100 that are radially displaced from the motor 110 and drive components housed therein. The engagement of corresponding alignment features on abrasion device 100 and skin attachment platform 200 aligns abrasion head 106 of abrasion device 100 with a target abrasion location on the skin in addition to holding the base 102 of abrasion device 100 in a stable, fixed position while shaft 104 is rotated.

Other exemplary embodiments of suitable skin attachment platforms or devices are illustrated in FIGS. 2B and 2C. In FIG. 2B, for example, skin attachment device 220 includes two or more skin attachment members 222a, 222b which are positionable on the skin surface about a target abrasion site. During use, attachment members 222a, 222b are designed to be placed on the skin surface spaced apart a distance 225 sufficient to enable the passage of a shaft of an abrasion device, such as abrasion device 100 (as well as an in vivo analyte sensor if one is used). Each member 222a, 222b includes a base or structure 224a, 224b, respectively, illustrated in the form of a block, which may be provided with an adhesive layer or pad 226a, 226b, respectively, to fix the bases 224a, 224b to the surface of the skin. Provided on a top surface of bases 224a, 224b, respectively, is one or more alignment features 228a, 228b, respectively, for mating engagement with alignment holes or the like of a skin abrasion device, such as abrasion device 100 (and optionally, an in vivo analyte sensor assembly if one is employed). Optionally, a second adhesive layer 230a, 230b may also be provided on the top surface of bases 224a, 224b, respectively, for facilitating attachment to an in vivo analyte sensor, described in greater detail below. One or both top adhesive layers 230a, 230b and bottom adhesive layers 226a, 226b may be replaceable or removable and be provided with releasable liners.

FIG. 2C illustrates another embodiment of a skin attachment device 240 having a similar configuration to that of skin attachment platform 220 of FIG. 2B. Skin attachment device 240 includes base structures 242a, 242b which are used in tandem to support, stabilize and align an abrasion device, such as abrasion device 100, at a target abrasion site on the skin surface. Respective adhesive pads 244a, 244b are employed to fix the attachment device to the skin surface and respective alignment features 246a, 246b are employed to engage with corresponding features of an abrasion device. In the illustrated embodiment, alignment features 246a, 246b are snap-fit alignment posts to provide additional fixed engagement with corresponding alignment holes within the base of an abrasion device.

Any of the subject skin attachment devices may include a mechanism or feature for controlling the depth of abrasion by a subject abrasion device. In certain embodiments, a frame, spacer or other structure may be mounted on either a bottom or top surface of the skin attachment device to increase the distance between the skin surface and abrasion head 106 when abrasion device 100 is operably mounted to a skin attachment device. As such, the depth to which skin may be abraded is thereby reduced. A plurality of such modular spacers of varying heights may be provided, where one or more are selectively mounted or removed from the skin abrasion device to provide the desired depth adjustment for a particular abrading application. The depth adjustment structure(s) may be removed after abrasion, or may be left in place. Alternatively, the skin attachment device may include an integral depth control mechanism, such as a telescoping sleeve, adjustable risers, or the like, which is selectively adjustable by the user. For example, the height of alignment posts 212, 228 and 246 (FIGS. 2A, 2B and 2C, respectively) may be adjustable by means of a control lever (not shown) accessible by the user. The height or depth adjustment mechanism, typically housed within the device body, may be mechanical or electronic, the latter requiring a power source, such as a battery, which is also housed within the device body. In certain embodiments, the depth adjustment feature may be configured to adjust the distance to which the abrasion head is extendable below the bottom surface of the skin attachment platform and, thus, controls the depth to which the skin is abraded.

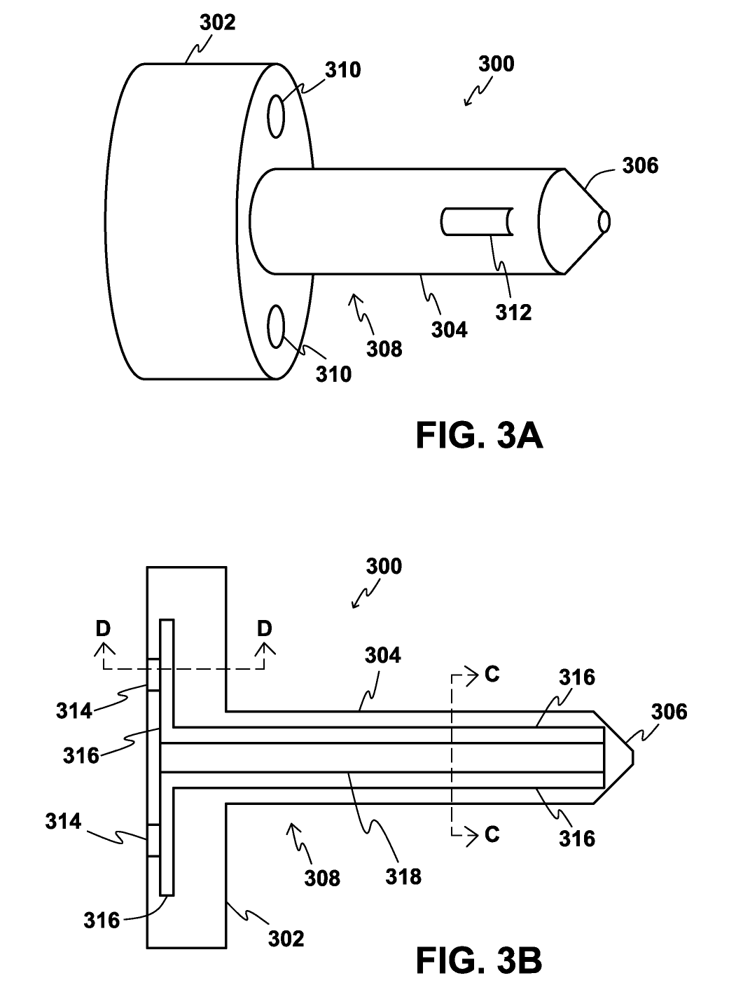

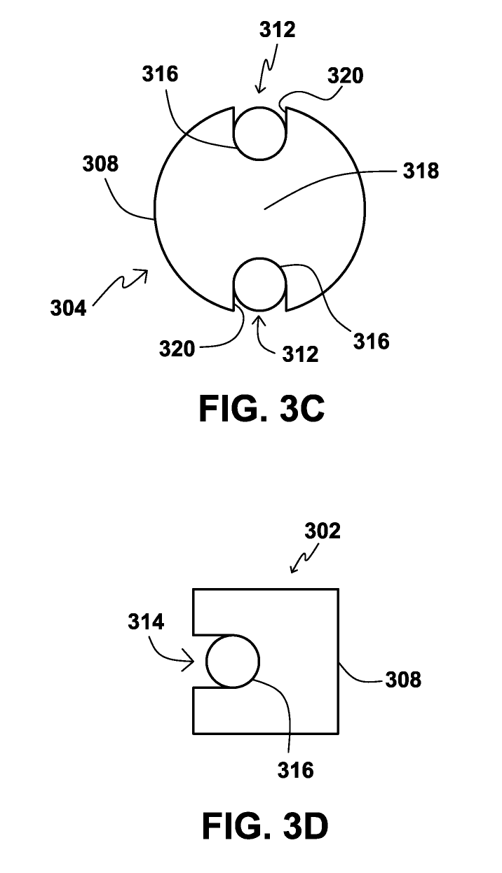

As briefly mentioned above, in addition to facilitating formation of abrasion sites on the skin, the subject skin attachment devices may be further configured to allow for the implantation of and the in vivo use of an analyte sensor within one or more of the formed abrasion sites. An exemplary in vivo sensor usable with the subject skin attachment devices and which is implantable within an abrasion site formed by the subject skin abrasion devices is described with reference to FIGS. 3A-3D.

As with the abrasion devices of the present disclosure, the in vivo analyte sensors of the present disclosure are needle-less or sharp-less, and are transcutaneously implantable without the use of a needle or sharp or other pointed implement and without cutting or lancing of the skin. As shown in FIG. 3A, in vivo analyte sensor 300 includes proximal portion 302 and distal portion 304. When sensor 300 is operatively transcutaneously implanted, sensor proximal portion 302 is positioned external to the skin surface and at least a distal tip 306 of distal portion 304 is positioned beneath the skin surface. In certain embodiments, such as the one illustrated, distal portion 304 has a substantially elongated construct and terminates distally in a blunted tip 306 to facilitate positioning within the skin or subcutaneous tissue. Tip 306 may be tapered, as illustrated, convex, hemispherical or the like. Sensor proximal portion 302 may have a structure which is substantially transverse to distal portion 304, is in substantially in the same plane as distal portion 304, or is juxtaposed at an angle to distal portion 304. Sensor proximal portion 302 may have a wider or broader construct than distal portion 304, or both portions may have the same general cross-sectional dimensions. The diameter of proximal portion 302 may be uniform or variable along its length, e.g., in a smooth or stepped transition fashion, and may range from about 3 mm to about 30 mm, typically from about 10 to about 20 mm. The diameter of distal portion 304 may be uniform or variable along its length, e.g., in a smooth or stepped transition fashion, and may range from about 0.5 mm to about 5 mm, typically from about 1 to about 3 mm. The length of distal portion 304 may range from about 1 mm to about 10 mm.

Sensor proximal portion 302 may include one or more alignment features 310, such as holes, similar to those described with respect to the subject abrasion devices above, to align and physically couple analyte sensor 300 with a skin attachment device or platform of the present disclosure in order to facilitate transcutaneous implantation of sensor 300 at a target in vivo site and to maintain the position of the implanted sensor for the useful life of the sensor or until the sensor is otherwise removed from the skin. In this embodiment, alignment features or holes 310 align and mate with alignment feature(s) on a skin attachment device or platform in a manner similar to that described above in which the abrasion devices engage with the skin attachment devices.

In certain embodiments, electrodes 316 and electrochemical sensing components of sensor 300 are wholly housed within a sensor housing, casing or body 308 made of a non-conducting or non-conductive material, such as plastic and the like. Plastics suitable for the sensor housing 308 include those approved for in vivo use by governmental regulatory agencies, including but not limited to PVC, silicones, polyurethane, poly(methyl methacrylate), and the like. Conductive materials are used to form the two or more electrodes 316, which comprise at least a working electrode and a reference or a counter/reference electrode of sensor 300. Additional electrodes may be used, e.g., for one or more additional working electrodes or separate reference and counter electrodes. The electrodes 316 are insulated from each other by an insulating material or structure 318, which may be an internal structure of housing 308 or may be a separate insulating material. As illustrated in FIG. 3A and FIG. 3C, sensor distal portion 304 includes at least one aperture 312 within housing 308 to expose the sensor electrodes 316 and electrochemical sensing material (not shown) to the ingress of bodily fluid, e.g., ISF, therethrough when the sensor is transcutaneously positioned and the apertures 312 reside within bodily fluid. Apertures 312 are positioned along a length of distal portion 304 that is implanted, and may be located within tip 306. As illustrated in FIGS. 3B and 3D, proximal ends of electrodes 316 are also partially exposed through one or more apertures 314 within sensor proximal portion 302 for electrically coupling to a sensor control or electronics unit (not shown) which may be releasably mounted to a subject skin attachment device for the duration of transcutaneous implantation of the sensor.

In certain embodiments, sensor housing/body 308 may be formed by molding a plastic material about electrodes 316, where the electrodes are exposed only at apertures 312 and 314 within the resulting sensor housing 308. Where a separate insulating material 318 is used, the electrodes 316 may first be formed or positioned on an insulating core 318 and the resulting structure is positioned in a mold to form the sensor housing 308 around the electrode-insulation core. For example, the latter embodiment may be fabricated by insert molding techniques. Alternatively, the electrodes 316 may be applied subsequently in channels 320 (see FIG. 3C) provided in the molded housing 308 by means of various deposition techniques e.g., chemical vapor deposition (CVD), physical vapor deposition, sputtering, reactive sputtering, printing, coating, ablating (e.g., laser ablation), painting, dip coating, etching and the like. Molding techniques as described herein provide a low-cost sensor manufacturing process that is highly repeatable and reliable. Examples of techniques which may be used to fabricate the subject sensors are described in, for example, U.S. Pat. Nos. 6,103,033 and 6,175,752.

The shape of the conductors that form the electrodes may be different from a cylindrical wire. For example, flat or curved pieces of metal or carbon could also be used. The diameters or widths of the electrodes may vary, but typically range from about 0.1 mm to about 3 mm, where in certain embodiments the diameter of a wire may be about 0.25 mm. The electrodes may be made of any suitable material, including but not limited to gold, palladium, platinum, silver, carbon, and a base metal such as nickel which may be plated with gold. Carbon need not be plated so it may be coated on a base metal using techniques such as electron beam deposition or the like. If a silver wire is used, prior to molding or positioning it into the sensor housing or body, the silver wire may be chloridized to form a silver chloride coating. The silver wire could also be chloridized after molding to form a silver/silver chloride (Ag/AgCl) coating.

The subject in vivo analyte sensors also include a sensing layer provided over at least the working electrode, collectively referred to at times as a sensing component, to make the sensor active towards the analyte(s) of interest, such as glucose. The sensing layer includes a chemical formulation to facilitate the electrochemical detection of the target analyte and the determination of its concentration in bodily fluid, particularly if the analyte cannot be electrolyzed at a desired rate and/or with a desired specificity on a bare electrode. A diffusion-limiting membrane may then optionally be provided over the sensing layer. The diffusion-limiting membrane is often beneficial or necessary for regulating or limiting the flux of analyte to the sensing layer. The diffusion-limiting membrane may also act as a mechanical protector that prevents the sensor components from leaching out of the sensor layer and reduces motion-associated noise.

The sensing layer and/or diffusion-limiting layers may be applied to the working electrode(s) in liquid form through aperture 312 of sensor housing 308 and allowed to dry. Aperture(s) 312 may include adherence-promoting features (not shown) in the form of ridges or roughness to help the dried layers adhere to the working electrode. In any case, in certain embodiments, the sensing and diffusion/limiting layers are completely contained within aperture 312 so that, during positioning of the sensor in vivo, the layers are protected from mechanical damage. Once the sensing and diffusion-limiting layers are applied to a sensor, the sensor may be sterilized and packaged, e.g., by e-beam or ethylene oxide sterilization.

Certain aspects and features of the subject analyte sensors may be fabricated according to sensor fabrication processes and techniques, including web-based manufacturing techniques, many of the steps of which are disclosed in U.S. Pat. No. 6,103,003 and U.S. Patent Application No. 2011/0021889, the disclosures of each of which are incorporated by reference herein.

It is noted that the present disclosure is also directed to in vivo sensors having multiple shafts or distal portions extending from a single base or proximal portion wherein each shaft houses a single electrode, e.g., a working a electrode. Each sensor shaft may having a sensing component configured to detect a particular analyte of interest where a plurality of analytes are simultaneously detectable by the single sensor implant, or all sensor shafts may be configured to detect the same analyte.

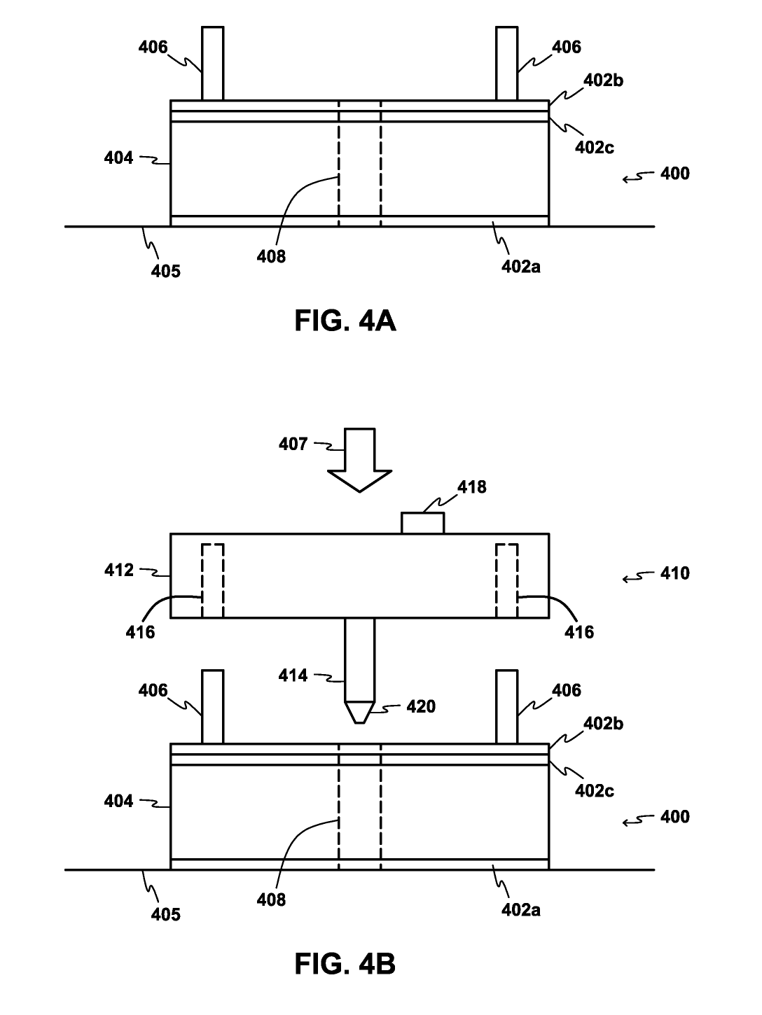

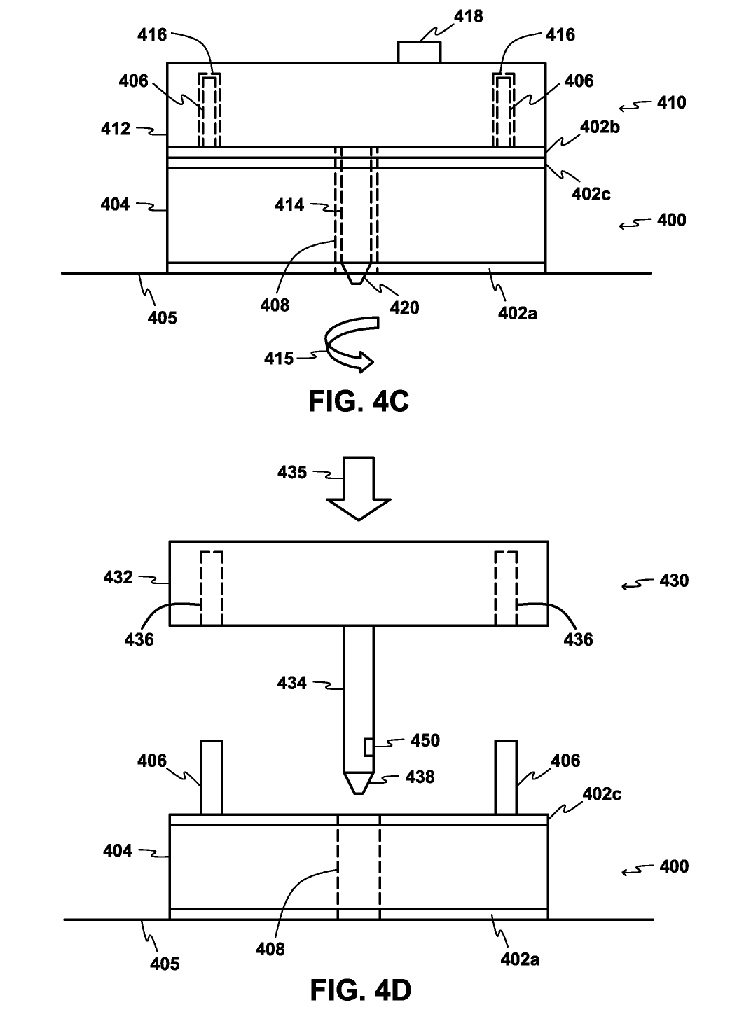

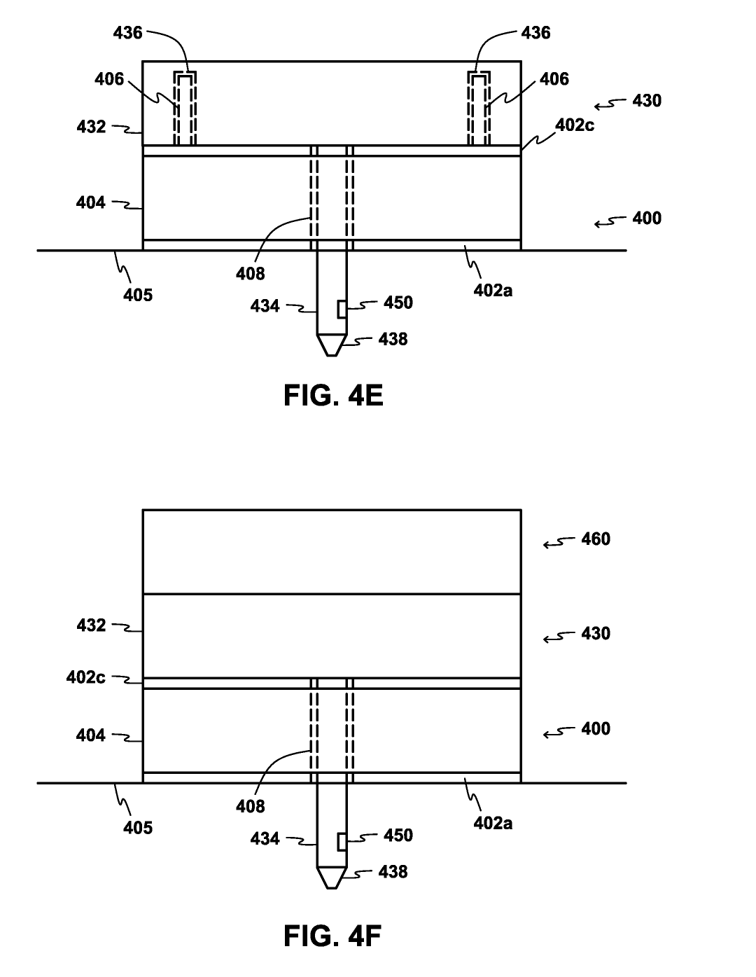

As mentioned above, accessing bodily fluid below the surface of the skin according to the methods and processes of the present disclosure may be for the extraction and collection of the bodily fluid for ex vivo analyte analysis or may be for the in vivo implantation of an analyte sensor. Referring now to FIGS. 4A-4F, examples of such methods and process are described which involve the use of one or more of the subject abrasion devices, skin attachment devices and/or in vivo sensors of the present disclosure. As previously mentioned, each of the subject abrasion devices and the subject in vivo analyte sensors may be used alone or in combination with a subject skin attachment device.

In certain embodiments, the methods of the present disclosure may include some or all of the following steps or techniques. Initially, one or more fluid collection or sensor implant sites are identified. Such target sites may include, but are not limited to, one or more the following body sites: forearm, upper arm, thigh and abdomen. Where ISF is being extracted for ex vivo testing, often a plurality of target abrasion/extraction sites may be necessary to provide the desired volume of ISF, for example, about 0.5 microliters. These target sites may be located within a few millimeters of each other, where the collective sites are often referred to as a “well”, or the target sites may be relatively spaced apart from each other, even on different body parts. If necessary, body hair at the target site(s) may be removed, and the site prepared using warm water and soap or an alcohol swab, for example.

As shown in FIG. 4A, attachment platform 400 is attached at the target site, e.g., at first target site, on the surface of the skin 405. In certain embodiments of skin attachment platform having an adhesive layer on a skin contacting surface thereof, the adhesive layer 402a is exposed, e.g., by peeling away a release liner, and the skin attachment platform 400 is firmly applied to the skin surface about the target abrasion site. In the embodiment of FIG. 4A, skin attachment platform 400 is of the variety of device 200 of FIG. 2A, having a central opening 408 within which the target abrasion site is aligned. If a skin attachment platform has more than one structural member, such as the skin attachment platform of FIGS. 2B and 2C, the structures are typically positioned on opposing sides of a target site. If employed, the depth adjustment feature (not shown) of the skin attachment platform is provided on the base structure 404 or otherwise adjusted to limit abrasion by the abrasion device to be used to the desired depth or to within the desired skin layer. The length of the abrasion shaft 414 and/or abrasion head 420 (FIG. 4B), and particularly the portion of that length which extends below the bottom surface of attachment platform 400, may vary depending on the skin type being abraded. For example, an individual with taut skin may require less extension than an individual with very loose skin.

As shown in FIG. 4B, abrasion device 410 is mated to attachment platform 400 (which, in certain embodiments, may be done prior to attaching attachment platform 400 to the skin surface) by aligning alignment holes 416 in the base 412 of the abrasion device 410 with alignment posts 406 of skin attachment platform 400, and then lowering abrasion device 410 in the direction of arrow 407, until it rests on top of skin attachment platform 400. This mating engagement has axially positioned shaft 414 of abrasion device 410 within the central opening 408 of attachment platform 400. Next, as shown in FIG. 4C, abrasion device 410 is activated by pressing activation button 418 which rotationally drives abrasion shaft 414 and thereby rotates abrasion head 420 against the skin surface 405 to abrade the skin. Depending on the depth adjustment made, head 420 abrades skin to a target skin layer, e.g., the stratum corneum, dermis, sub-dermal layer, etc. While ISF may be accessed in each of the stratum corneum, epidermis and dermis, as nerve endings and capillaries are located in the dermal layer, formation of an opening through the stratum corneum and into the epidermis but which does not reach the dermis has the benefits of being painless and bloodless.

The abrasion process may be repeated as necessary to form a plurality of contiguous or non-contiguous target abrasion spots, for example, two, three, or four or more. For ISF extraction applications, the number of abrasion sites may depend on the necessary volume of ISF that is extractable from each site. For in vivo sensing applications, the number of abrasion sites will depend on the number of sensors or sensor shafts to be implanted. As discussed above, the formation of multiple skin abrasion sites may involve the removal, repositioning and remounting of an abrasion device relative to an attachment device where the abrasion head is aligned with an additional target site on the skin surface. Repositioning the abrasion device may involve axially rotating or laterally repositioning it relative to the skin attachment member. Still yet, the formation of additional bodily fluid access or sensor implant sites may involve the collective repositioning of both devices to a different body location. Each abraded site may have a diameter of about 1 mm to about 2 mm. If multiple spots are created, some or all may be co-located but separated from each other by no less than about 2 mm to about 3 mm.

The depth of abrasion may depend on the type of bodily fluid being targeted and whether the application is for ex vivo or in vivo analyte testing. For applications in which ISF is the target fluid, the skin is abraded at least through the stratum corneum and may extend into the epidermis, which depth varies from host to host. As such, the depth of abrasion (i.e., the depth below the skin surface) ranges from at least about 0.02 mm to about 0.12 mm, and more typically from about 0.06 mm to about 0.10 mm to ensure penetration into the epidermis but not to within the dermis layer to avoid bleeding. In other applications, e.g., those in which the dermis is the target skin layer, the depth of abrasion may be from about 0.1 mm to about 2.0 mm or greater, depending on the individual.

With respect to the rotational speed (revolutions per minute) of the abrasion head 420, this setting must be sufficiently high to rapidly remove the skin, but must also be sufficiently low to avoid an excessively short application time that would be difficult to control. The duration of abrasion must be sufficiently high to ascertain that the proper depth is reached but must be sufficiently low that abrasion is only to the target skin layer. Rotation may be continuous or pulsed, i.e., discontinuous, in certain embodiments. The speed may be adjustable or fixed, and may be a constant (i.e., a single speed) or non-constant (i.e., two or more speeds) during the course of a given abrasion process. The speed of rotation of the abrasion head or grindstone may range from about 1,000 rpm to about 25,000 rpm, e.g., about 5,000 rpm to about 10,000 rpm; and the abrasion time may be less than about 1 second in certain embodiments, or may range from about 1 second to about 3 minutes, e.g., from about 15 seconds to about 45 seconds. Experiments with humans have shown that higher speeds for shorter times, rather than slower speeds for longer times, are better at opening the skin without causing bleeding, i.e., removing the stratum corneum without going into the dermis.

For ISF extraction applications, once abrasion of the skin is completed, abrasion device 410 is typically removed from skin attachment platform 400. Confirmation of access to ISF may be achieved by direct visual observation and/or under magnification, and/or by expression or suction of a few microliters of fluid from the site. Once the ISF extraction site or well has been prepared, the skin attachment platform 400 may also be removed. In certain embodiments, if the extraction means, e.g., vacuum, heat, etc., is integrated within abrasion device 410, those means are activated to retrieve the desired volume of ISF while abrasion device 410 is still operatively mounted to attachment platform 400 (with abrasion head 420 retracted so as not to interfere with the flow of fluid from the abrasion site). If not integrated within device 410, the separate fluid extraction mechanism (not shown) is then used, without or without the use of the skin attachment device 400. If vacuum is used for the ISF extraction, a pressure of down to about −14.7 psig, and more typically from about −3.0 psig to about −10.0 psig is used. The expressed ISF may be collected into capillary collection tubes for later analysis and/or applied directly to in vitro analyte test strips, such as FreeStyle® blood glucose test strips by Abbott Diabetes Care Inc. when the analyte of interest is glucose.

For in vivo analyte monitoring applications, once abrasion of the skin is completed, abrasion device 410 is removed from skin attachment platform 400. Depending on the embodiment used, abrasion head 420 or both the abrasion head 420 and abrasion shaft 414 are discarded, or otherwise sterilized for later reuse, and base 404 is retained (if not intended to be disposable) for later use. The depth adjustment structure (not shown), if separable from base structure 404 and if not necessary for controlling the depth of implantation of the analyte sensor may be removed. Next, release liner 402b is removed from the top side of skin attachment platform 400 to expose a second adhesive layer 402c (see FIG. 4D). An analyte sensor 430 is then operatively mounted to skin attachment platform 400 by aligning alignment holes 436 of the sensor proximal portion 432 with alignment posts 406 of skin attachment platform 400 and lowering the sensor 430 onto the skin attachment platform 400 in the direction of arrow 435, as shown in FIG. 4D. In so doing, sensor distal portion 434 is axially positioned within opening 408 of attachment platform 400, and axially aligned atop an abraded site on the skin surface. With (at least) the stratum corneum removed at the insertion site, little resistance is offered against penetration of the blunt distal end 438 of sensor distal portion 434 into the abrasion site previously created in the skin by abrasion device 410, as shown in FIG. 4E, to a depth of about 1 mm to about 10 mm below the skin surface 405. In particular, the portion of sensor distal portion 434 that includes aperture(s) 450 is positioned below the surface of the skin 405 to expose the internally housed electrodes to the targeted bodily fluid. The blunt sensor distal end 438 causes little damage to the skin layers and subcutaneous space because it is not sharp enough to cut blood vessels and, thus, reduces the likelihood of any bleeding. Thereafter, sensor 430 is securely retained to attachment platform 400 by adhesive 402c for a period of time that may range from about one hour to multiple days or more, e.g., about one week or more, e.g., about ten to about fourteen days or more, e.g., about multiple weeks or more such as about one month or more.

In certain embodiments, as shown in FIG. 4F, a sensor control or electronics device or unit 460 is operatively coupled to sensor proximal portion 432 by means of a snap-fit engagement (not shown) and/or other attachment feature(s). That is, electronics unit 460 makes electrical and physical contact with the sensor electrodes through apertures within the sensor proximal portion, as described previously with respect to FIGS. 3A-3D. In certain embodiments, however, the electronics are integrally and permanently coupled to the sensor to form a single unit. Electronics unit 460 houses data processing, data storage and communication electronics, the latter of which may include a transmitter for relaying or providing data obtained using the sensor to another device such as a remotely located device, and may also include a variety of optional components, such as, for example, a receiver, a power supply (e.g., a battery), an alarm system, a display, a user input mechanism, a data storage unit, a watchdog circuit, a clock, a calibration circuit, etc. The data processing electronics measure an electrochemical reaction occurring at the sensor electrodes and may be configured to display or provide such results directly to the user, or alternatively or additionally, may be configured to forward data related to the measured reaction to a spaced apart receiver unit (not shown) where the results may be formatted for display to a user. The receiver unit may be electrically coupled to electronics unit 460 with a wired or wireless connection, e.g., radio frequency (RF), infrared (IR), or the like.

Upon expiration of the useful life of the implanted in vivo analyte sensor 430 or when the user has finished analyte monitoring, electronics unit 460 may be separated from sensor 430, and the sensor and skin attachment platform 400 are removed simultaneously or serially from the skin 405. In certain embodiments, electronics unit 460 is not separated from sensor 430 for removal, and/or electronics unit 460 and sensor 430 are removed from the user simultaneously, although they may be removed serially. Electronics unit 460 may be a re-useable component in certain embodiments, e.g., where it is separable from a sensor. Examples of electronics units that may be employed with embodiments of the subject present disclosure are described, for example, in U.S. Pat. No. 6,175,752, the disclosure of which is herein incorporated by reference.

The subject systems may be configured to enable a plurality of abrasion sites to be made on the skin surface. For example, an abrasion device 100 may be configured to be mountable to a single skin attachment platform at a plurality of selective positions or orientations. As such, the skin attachment platform need only be applied once and affixed at a single location on the skin, while the abrasion device is mounted and dismounted from the skin attachment device any number of times, depending on the number of abrasion sites to be formed. To enable such functionality, a skin attachment device may have a plurality of openings or spaces within which to operatively align a rotatable shaft of a subject abrasion device.

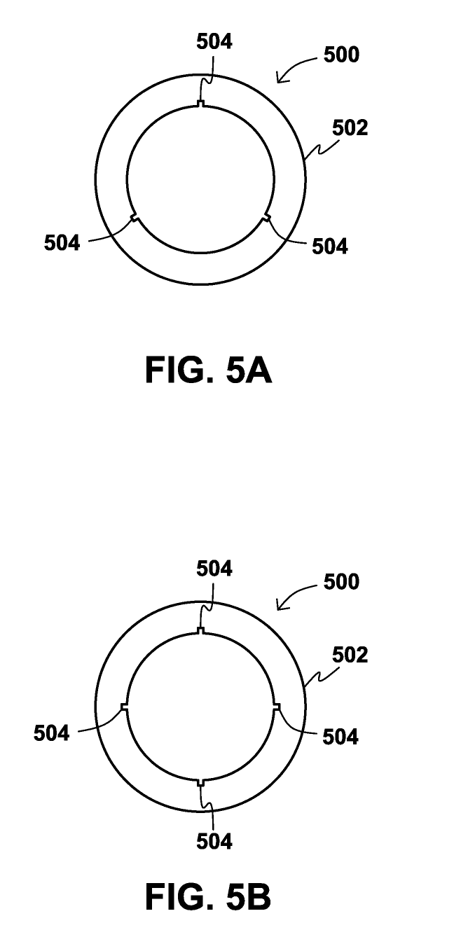

In certain embodiments, a skin attachment platform may have a plurality of alignment features which enable variable rotational positioning of an abrasion device about the skin attachment member, such that the abrasion shaft and head are selectively positionable at a plurality of locations on the skin surface. For example, as shown in FIGS. 5A and 5B, a skin attachment device 500 having an annular base 502 with a general configuration similar to that of skin attachment platform 200 of FIG. 2A has a plurality of alignment features 504 which are mateable with corresponding alignment features of an abrasion device (not shown). In certain embodiments, the abrasion shaft of the abrasion device is not centrally positioned, as it is in abrasion device 100, but radially displaced from the central axis such to enable selective rotational positioning of the abrasion device by varying the positions 504 about skin attachment device 500. In other embodiments, the shaft is centrally positioned, however, the base of the abrasion device is shaped or configured to provide lateral displacement of the shaft when positioned at the various positions provided by alignment features 504. While FIGS. 5A and 5B respectively illustrate skin attachment devices having three alignment features 504 (enabling three abrasion sites rotationally displaced from each other about 120°) and four alignment features 504 (enabling four abrasion sites displaced from each other about 90°), any number of alignment features may be provided at any number of locations about base 502, where the positioning of such features 504 may be symmetrical or asymmetrical. Further, while the illustrated skin attachment devices are configured to enable one or more abrasions to be made within a central opening of the device bases, other embodiments may be configured to enable attachment of an abrasion device such that the abrasions may be made on skin positioned on the outside or perimeter of the base structure of the attachment device. Accordingly, the plurality of skin abrasion sites may be provided without moving the skin attachment member from a single initial location on the skin. The provision for multiple abrasion sites may be convenient if more than one discrete abrasion area is desired, e.g., for the contemporaneous implantation of more than one in vivo sensor or in order to extract a sufficient volume of ISF to conduct a particular ex vivo assay. As such, the present disclosure includes systems or kits which include an abrasion device with one or more skin attachment devices or members which enable selective abrasion of a plurality of target skin sites.

While the present disclosure has been described with reference to the specific embodiments thereof, it should be understood by those skilled in the art that various changes may be made and equivalents may be substituted without departing from the true spirit and scope of the present disclosure. In addition, many modifications may be made to adapt a particular situation, material, composition of matter, process, process step or steps, to the objective, spirit and scope of the present disclosure. All such modifications are intended to be within the scope of the claims appended hereto.

What is claimed is:

1. A method of transcutaneously positioning a sensor, the method comprising:
abrading a target site on a skin surface to a selected depth; and
positioning an implantable portion of a sensor into the skin through the abraded target site.

2. The method of claim 1 wherein the selected depth is not greater than the epidermis layer of the skin.

3. The method of claim 2 wherein the selected depth is not greater than the stratum corneum layer of the skin.

4. The method of claim 1 wherein the abrading the target site comprises using an abrasion device having a blunted skin-contacting surface.

5. The method of claim 1 wherein the inserting the sensor into the skin is performed without using an introducer.

6. The method of claim 1 wherein the sensor comprises a blunted skin-penetrating end.

7. The method of claim 1 further comprising identifying two or more target sites on the skin surface and repeating the abrading and sensor positioning steps for each target site.

8. The method of claim 1 wherein the sensor is an analyte sensor.

9. The method of claim 1 wherein the sensor is an electrochemical sensor.

10. The method of claim 1 further comprising, prior to abrading the target site, affixing a platform to the skin surface adjacent the target site and mounting an abrasion device to the platform.

11. The method of claim 10 further comprising adjusting the height of the platform to selectively control the depth of abrasion by the abrasion device.

12. The method of claim 10 wherein affixing the platform to the skin surface comprises providing an adhesive layer.

13. The method of claim 10 further comprising mounting a sensor control unit to the platform and to the sensor after transcutaneously positioning the sensor.

14. The method of claim 1 further comprising selectively adjusting the depth of abrasion.

15. The method of claim 1 wherein a distal end of the sensor is positioned to a depth below the epidermis layer of the skin.

16. The method of claim 1 wherein a distal end of the sensor is positioned within the epidermis layer of the skin.

17. The method of claim 1 further comprising selectively controlling the speed of abrasion.

18. The method of claim 1 wherein the positioning of the sensor comprises aligning the sensor with the abraded target site.

19. A method of transcutaneously accessing bodily fluid, the method comprising:
identifying one or more bodily fluid access sites on a skin surface;
affixing at least one skin attachment platform to the skin surface adjacent at least one bodily fluid access site;
mounting an abrasion device comprising an abrasion head having a blunted skin-contacting surface to the at least one skin attachment member wherein the abrasion head is aligned above the bodily fluid access site; and
abrading the skin surface to a selected depth at the bodily fluid access site.

20. The method of claim 19 further comprising adjusting the height of the platform to selectively control the depth of abrasion by the abrasion device.

21. The method of claim 19 further comprising selectively controlling the depth of abrasion.

22. The method of claim 19 wherein the selected depth is not greater than the epidermis layer of the skin.

23. The method of claim 22 wherein the selected depth is not greater than the stratum corneum layer of the skin.

24. The method of claim 19 further comprising extracting a volume of bodily fluid from the bodily fluid access site.

25. The method of claim 24 wherein the bodily fluid includes interstitial fluid.

26. The method of claim 24 wherein the extracting a volume of bodily fluid comprises one of more of applying negative pressure to the skin, mechanically stretching the skin, applying capillary action to the skin, heating the skin, or applying a chemical at the at least one bodily fluid access site.

27. The method of claim 19 further comprising:
remounting the abrasion device to the at least one skin attachment member wherein the abrasion head is aligned above a second bodily fluid access site; and
abrading the skin surface to a selected depth at the second bodily fluid access site.

28. The method of claim 27 wherein the remounting the abrasion device comprises axially rotating the abrasion device relative to the skin attachment member.

29. The method of claim 27 wherein the remounting the abrasion device comprises laterally repositioning the abrasion device relative to the skin attachment member.

30. A transcutaneously implantable sensor comprising:
a proximal portion configured for positioning above a skin surface and for connecting with a sensor control unit; and
a distal portion having a blunted distal end configured for penetrating through an opening within the stratum corneum layer of the skin.

31. The sensor of claim 30 wherein the distal portion includes an implantable length sufficient for extending into subcutaneous tissue when the sensor is operatively transcutaneously positioned.

32. The sensor of claim 31 further comprising at least one aperture within the implantable length to expose a sensing component within the distal portion.

33. The sensor of claim 32 wherein the sensing component comprises a portion of a working electrode.

34. The sensor of claim 30 further comprising at least one electrode extending from within the distal portion to within the proximal portion.

35. The sensor of claim 30 wherein the distal portion comprises a substantially elongated configuration.

36. The sensor of claim 30 wherein at least the distal portion comprises a non-conductive housing.

37. The sensor of claim 36 further comprising at least one electrode formed within the housing, wherein the housing comprises an aperture for exposing the at least one electrode to bodily fluid when the distal portion is positioned through the skin.

38. The sensor of claim 30 wherein the proximal portion comprises at least one feature for matingly engaging with a platform configured for mounting to the skin surface.

39. The sensor of claim 38 wherein the platform comprises at least one alignment feature for aligning the sensor with the opening in the strateum corneum layer of the skin.

40. A method of fabricating a transcutaneously implantable sensor, comprising:
providing at least one conductor;
forming a sensor housing made of non-conductive material about the at least one conductor, wherein the housing comprises a proximal portion and a distal portion, the proximal portion configured for positioning above a skin surface and for electrically connecting with a sensor control unit, and the distal portion having a blunted distal end configured for penetrating through an opening within the stratum corneum layer of the skin; and
forming at least one aperture within an implantable portion of the distal portion of the housing, the aperture configured for exposing the at least one conductor therethrough.

41. The method of claim 40 further comprising providing the at least one conductor on an insulating core, wherein forming the sensor housing comprises molding the non-conductive material about the insulating core.

42. The method of claim 40 wherein the forming the sensor housing and the forming the at least one aperture are performed simultaneously by means of a molding process.

43. A method of fabricating a transcutaneously implantable sensor, comprising:
providing a sensor housing made of non-conductive material, wherein the housing comprises a proximal portion and a distal portion, the proximal portion configured for positioning above a skin surface and for electrically connecting with a sensor control unit, and the distal portion having a blunted distal end configured for penetrating through an opening within the stratum corneum layer of the skin;
forming at least one channel within the sensor housing wherein the at least one channel extends from within the distal end of the distal portion to within the proximal portion;
providing at least one conductor within the at least one channel; and
forming at least one aperture within the distal end of the distal portion of the housing, the aperture configured for exposing the at least one conductor therethrough.

44. A system for abrading a skin surface, the system comprising:
an abrasion device comprising a base unit and a rotatable shaft having an abrasion head provided at a distal end thereof; and
a skin attachment platform comprising a bottom surface configured for placement on a skin surface and a top surface configured for releasably mounting the abrasion device thereto, the skin attachment platform further comprising a component for adjusting the depth to which the abrasion head is extendable below the bottom surface of the skin attachment platform when the abrasion device is mounted thereto.

45. The system of claim 44 wherein the bottom surface of the skin attachment platform comprises an adhesive.

46. The system of claim 44 wherein the base unit includes a component for rotating the shaft at a selectable speed.

47. The system of claim 44 wherein the depth adjustment component comprises height-adjustable posts provided on the top surface of the skin attachment platform.

48. The system of claim 44 wherein the depth adjustment component comprises at least one removable spacer layer positionable on the top surface of the skin attachment platform.

49. The system of claim 44 wherein the abrasion device includes a plurality of interchangeable abrasion heads.

50. The system of claim 49 wherein each of the abrasion heads is unique from the other abrasion heads in one or more of shape, size and abrasiveness.

51. The system of claim 44 wherein the skin attachment platform comprises at least two cooperative structures.

52. The system of claim 44 wherein the abrasion head is comprised of material effective in abrading the stratum corneum and ineffective in abrading the epidermis layers of skin.
